(19) United States
(12) Patent Application Publication
(10) Pub. No.: US 2006/0068408 A1
(43) Pub. Date: Mar. 30, 2006

(54) Method for determining DNA damage and multi-chamber plate

(76) Inventors: Irene Witte, Oldenburg (DE); Hartmut De Wall, Oldenburg (DE)

Correspondence Address: WILLIAM COLLARD;COLLARD & ROE, P.C., 1077 NORTHERN BOULEVARD, ROSLYN, NY, 11576, US

(21) Appl. No.: 11/069,752

(22) Filed: Mar. 1, 2005

(30) Foreign Application Priority Data
Sep. 24, 2004 (DE) 10 2004 046 364.6

(51) Int. Cl. C12Q 1/68

(52) U.S. Cl. 435/6

(57) ABSTRACT

In a method for determining DNA damage, wherein cells are treated with genotoxic agents and lysed and DNA fragments of the cells are separated according to their length by a physical separating method, cells are put into various chambers of a multi-chamber plate and a genotoxic chemicals are put into the various chambers of the multi-chamber plate. The cytotoxicity is then measured and the walls of the multi-chamber plate are then removed and the physical separation process is carried out together for all samples. The multi-chamber plate has removable walls and at least one coating in the bottom region. Samples for testing for DNA damage can be investigated especially quickly, effectively and inexpensively by the method using the coated multi-chamber plate.

### BACKGROUND OF THE INVENTION

1. Field of the Invention

The invention relates to a method for determining DNA damage, wherein cells are treated with genotoxic agents and lysed and DNA fragments of the cells are separated according to their length by a physical separating method. Cells are put into various chambers of a multi-chamber plate and the chemical solutions are put into the various chambers of the multi-chamber plate. The invention further relates to a multi-chamber plate for biological, medical, pharmaceutical and chemical research, especially for implementing the method.

2. The Prior Art

One method for determining DNA damage is the so-called “comet assay”. This method is used to determine DNA damage in individual cells. In this case, the DNA damage is detected as DNA strand breaks. It is also possible to determine the persistence of the damage, i.e., the repair of the DNA damage.

For implementing the conventional “comet assay” (also called “single cell gel electrophoresis”), adherently growing cells are put into a Petri dish and the cells adhering to the bottom of the culture vessel are incubated with the genotoxic agents. The cells are then released (trypsinated) from this Petri dish, mixed with LMP agarose and transferred to a slide pre-coated with agarose. There, the cells are lysed (cell membranes are dissolved) and the DNA is released. After an alkaline treatment the DNA fragments are separated according to their length by means of a physical separating method, electrophoresis. In this case, the negatively charged DNA molecules migrate within a carrier material according to their length. The DNA of each individual cell can be made visible under a fluorescence microscope after staining. The undamaged DNA appears as a round spot (so-called comet head). In the case of DNA fragmentation, as a result of damaged DNA, a more or less marked tail additionally appears (a so-called comet tail) with a simultaneous reduction in the intensity of the comet head. The intensity or brightness of the tail depends on the DNA migration from the nucleus region. The longer the tail, the smaller the fragments formed. As a quantifiable measure of the DNA damage, either a classification into classes of damage is made or the tail intensity or a product of the percentage fraction of DNA in the tail and the tail length is given (the so-called tail moment).

### SUMMARY OF THE INVENTION

It is an object of the invention to simplify the implementation of the method for the investigation of a plurality of samples and to make it possible to study a plurality of samples quickly.

In the method according to the invention, cells are treated with genotoxic agents and lysed, and DNA fragments of the cells are separated according to their length by a physical separation process, wherein cells are put into different chambers of a multi-chamber plate and treated with genotoxic agents. It is important according to the invention that the walls of the multi-chamber plate are removed and that the physical separation process is carried out for all the samples together without previous trypsination of the cells. As a result, a plurality of samples can be investigated in parallel and this can be carried out largely automatically. The investigation expenditure is thereby reduced considerably and the number of investigation steps is decisively shortened by omitting the step of trypsination of the cells. Compared with known techniques, the number of samples studied can be increased from around 12-24 to 300-400 per day per person. The physical separation method, and in particular the electrophoresis, is carried out together and at the same time for all the samples. The method is especially suitable for adherent cells. A use for non-adherent cells or cells in solutions is also feasible.

It is especially preferred if the chemical treatment of the cells and the separation method take place in the same place. Thus, the cells need not be trypsinated and transferred. In a preferred further embodiment of the invention, the chemical treatment of the cells is carried out in parallel,. Multi-pipettes or robots can be used for this purpose so that a test substance or test substance mixture is simultaneously put into each chamber of the multi-chamber plate. In a further preferred embodiment, the lysis of the cells takes place after removing the chamber walls. This means that the lysing step is only carried out after removing the walls. It is also feasible to carry out this step before removing the walls.

In a preferred further development of the invention, after the physical separation of the DNA, the treated cells are moved away with a microscope displaceable in at least two dimensions and the images determined are evaluated. This preferably takes place fully automatically so that this can take place overnight and thereby approximately 50 working hours can be saved. In this case, the microscope is preferably fitted with a mechanical stage adjustable in the x, y and z direction, that is displaceable in three dimensions, and fitted with a control device for moving away the plurality of samples and has software which makes it possible to sharply adjust the microscope to the comets. The microscope picture is then photographed and an automatic evaluation of the comets takes place, preferably via the classification into pollutant classes or via the tail length or via the product of the percentage fraction of DNA in the tail and the tail length. Furthermore, it is also possible to automatically represent these results graphically.

In a further development of the method, a toxicity test is also incorporated into the method. The toxicity test preferably takes place on the same cells for which the DNA damage is measured. The FDA assay is preferably used for the toxicity investigation. The vitality of the cells after chemical treatment in the multi-chamber plate before the cell lysis can be measured on the basis of the fluorescein diacetate toxicity test (FDA test) according to Rotman and Papermaster (Proc. Nat. Acad. Sci. USA 55, 131-141). In this test, damaged cells are reduced in their capability to metabolise FDA to fluorescent fluorescein. The fluorescence intensity is measured using a fluorescent plate reader. The chamber walls are then removed and the cells are lysed and the necessary steps are carried out for the electrophoresis. As a result of the high sensitivity of the FDA test, a small number of cells is sufficient for the method to arrive at a statistically secured prediction of toxicity. The side walls are constructed as black for better implementation of the fluorescence measurement.

A further aspect of the invention consists in providing a multi-chamber plate for biological, medical, pharmaceutical and chemical investigations, especially for implementing the method described above, wherein this multi-chamber plate is distinguished by the fact that it has at least one coating in the bottom area and the walls of the multi-chamber plate are removable. The multi-chamber plate is especially suited for the simultaneous investigation of various samples. Preliminary investigations such as cytotoxocity studies can first be carried out in the individual chambers and after removal of the walls the treated cells remain adhered to the coating and can be simultaneously and automatically further processed, especially using a physical separating method, especially electrophoresis. The bottom of the multi-chamber plate, especially each bottom area of each chamber is preferably constructed as planar in order to simplify the further investigations. Good cell adhesion with the coating with a rounded cell shape is achieved. As a result of the coating, trypsination is no longer necessary and the cells do not need to be transferred.

In order to configure the walls of the multi-chamber plate as removable, these are preferably affixed to the bottom of the multi-chamber plate using an adhesive compound or sticking compound. This form of fixing forms a type of preset breaking point. In another further development, the walls are mechanically affixed to the bottom via the preset breaking point using a plug connection or a detachable locating connection.

The multi-chamber plate can be constructed in different sizes. The construction of a multi-chamber plate with 96 chambers is preferred. In other embodiments, such a multi-chamber plate has eight chambers. Other sizes can also easily be produced, for example, 6, 12, 24, 48, 192 or almost any other sizes. The walls of the multi-chamber plate are transparent for the detection of pure DNA damage and for co-detecting the cytotoxicity are preferably constructed as non-reflecting, especially black. In this way a fluorescein test can also be carried out in the chambers of the multi-chamber plate.

In another preferred further development of the invention, the multi-chamber plate is coated in the bottom area with a carrier material for conducting a physical separation method, especially electrophoresis. In this way, the cells inserted in the multi-chamber plate can also remain in the multi-chamber plate after chemical treatment and be lysed after removing the side walls. The physical separation method then can also be carried out directly on these samples. The carrier material preferably has a mixture of gelatin and agarose. This coating ensures that the rounded cell shape is retained, the cells do not spread out and need not be trypsinated. The greater fraction of the mixture is preferably gelatin. The mixture ratio of agarose to gelatin is preferably 1:10 to 1:2, especially 1:5 to 1:3 and especially preferably 1:3.3.

In a further preferred embodiment of the invention, a base layer, especially agarose, is provided underneath the carrier material. This serves, among other things, for better adhesion of the aforesaid carrier layer. The agarose in the base layer also contributes towards better implementation of the electrophoresis. Various types of agarose in various concentrations can be used. Alternatively, the base coating can also be constructed such that the bottom of the multi-chamber plate is roughened and in this way, better adhesion of the carrier material is achieved.

In a further development of the invention, a further layer is provided above the carrier layer. This serves to improve the adhesion conditions of the cells and is particularly important for carrying out the toxicity assay. This cell adhesion layer is preferably poly-lysine. Especially used for this purpose is poly-D-lysine or poly-L-lysine, especially in a concentration of around 50 μg/ml. It is also possible to use fibronectin, especially in concentrations of 0.05 to 0.01 mg/ml, collagen, especially in concentrations of 0.1% to 0.5% (W/V) or a thin film of a fetal calf serum. These additives can also contain calcium and magnesium ions. The coating of the multi-chamber plate preferably takes place before implementing the method. The coating with LMP agarose takes place after dismantling the multi-chamber plate.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

FIG. 1 shows a perspective view of a multi-chambered plate according to the invention; and

FIG. 2 shows a perspective view of the multi-chambered plate of FIG. 1 with the walls removed.

### DETAILED DESCRIPTION OF THE PREFERRED EMBODIMENTS

The implementation of the entire method is explained subsequently with reference to a preferred exemplary embodiment and the drawings.

The preferred specification for implementing the combined cytotoxicity/gene toxicity experiment (FDA determination /comet assay) for adherent cells is as follows:

Sew cells in the MCW (final concentration: dependent on chamber size)

Incubate for 15 minutes to 2 hours with the genotoxic chemical(in serum-free medium) in various concentrations

Remove genotoxic solutions

For 5-10 min. 50 μl FDA solution: 60 μl stock solution (50 mg FDA in 10 ml acetone) incubate in 10 ml PBS buffer/chamber in the dark

Measure in fluorescence plate reader (excitation: 485 nm, emission: 520 nm)

Dismantling the walls of the mcp (multi-chamber plate). All the following steps to be carried out in subdued light

Dip the dismantled mcp in dissolved 37 [symbol] C warm IMP agarose (0.25 g IMP agarose in 50 ml PBS buffer)

Leave agarose layer to cool for 3-5 min at 3 [symbol] C

Incubate for 1 h in freshly made lysis solution (for composition see below) at 0 [symbol] C

Put mcp plate in horizontal electrophoresis chamber and leave for 40 min in ice-cold alkaline electrophoresis buffer (for composition see below) (alternatively the neutral comet assay can also be carried out to determine double strand breaks).

Carry out electrophoresis for 20 min at 25 V and 300 mA

Take mcp plate from the electrophoresis chamber and place for 5 min in neutralisation buffer (48.5 g/l Tris, adjust to pH 7.5 with fuming HCl); repeat process 2×

Rinse briefly with doubly distilled water

Dry mcp plate (at 20° C.)

Stain samples, for example with ethidium bromide solution (2 μg/ml)

Cover mcp with a cover glass (in the size of the mcp) and after 15 min evaluate the comets fully automatically (excitation point 515-560 nm, barrier filter 590 nm)

Coloration can be conserved over a fairly long time by using antifade solution

Solutions:

LIMP agarose:
0.250 g LIMP agarose (Sea Plaque GTG agarose)
50 ml PBS buffer
dissolve by boiling in the microwave,
apply to the mcp at 37° C.
   Lysis Solution:
Stock solution:
2.5 M NaCl
100 mM EDTA (Titriplex III)
10 mM Tris
Dissolve salts in 8 g/l NaOH and adjust pH to 10
1% Na lauryl sarcosinate (10 g)
fill up to 890 ml with doubly distilled water
   Solution for Use:
1 ml Triton X-100
10 ml DMSO
89 ml lysis stock solution
   Electrophoresis Buffer:
Stock solutions:
a) 10 mol/l NaOH (in doubly distilled water)
b) 0.2 mol/l EDTA (in doubly distilled water) adjust to pH 10
   Solution for Use:
30 ml a)+5 ml b), fill up to 1 l doubly distilled water, pH should be >13.
   Formulation for Coating the mcp:

The coating thickness should be around 50 μm in total

1st Coating:

Place 3 g of Seakem agarose LE in 200 ml PBS buffer (Dulbecco's)

Boil briefly and cool to 60° C.

Repeat boiling and cooling procedure 3×

Coat (dip) surface at 100° C. and leave to dry

2nd Coating:

Dissolve 5 g of gelatin in 500 ml doubly distilled water at 60° C.

Mix 3.3 parts gelatin solution with 1 part ready made agarose solution and heat to 100° C.

Apply to first coating layer (dip) at 100° C. and leave to dry

3rd Coating:

Apply 50 μg/ml of poly-L-lysine in doubly distilled water to the second coating layer

Incubate for 30 min in incubator at 37° C.

Rinse with PBS buffer (Dulbecco's)

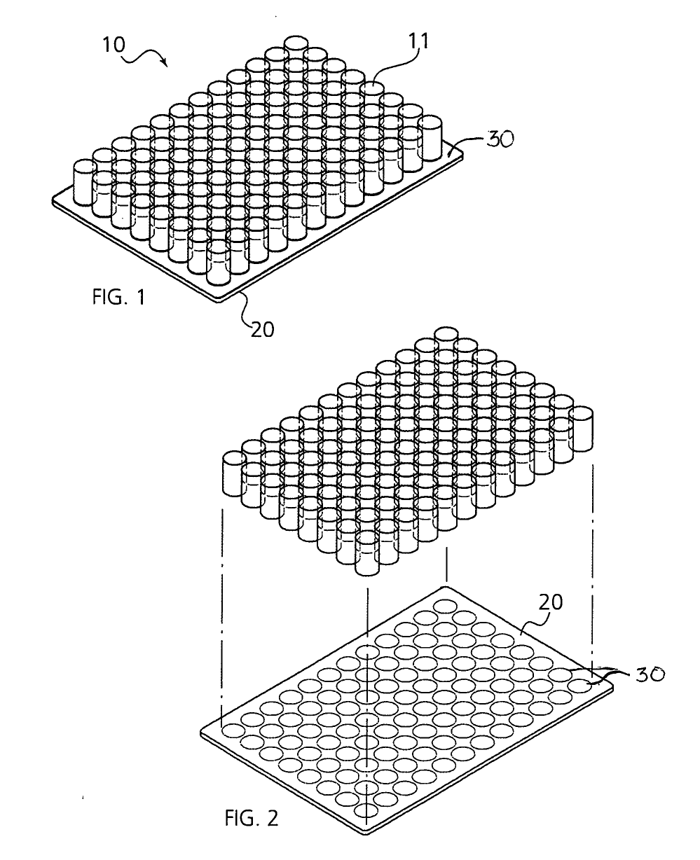

One example of a plate used in the method according to the invention is shown in FIGS. 1 and 2. This plate 10 comprises a plurality of chambers 11, which are removable from bottom 20, so as to allow the contents in each chamber to mix after the treating step and for the physical separation to take place for all chambers at once. Furthermore, the bottom 20 of the plate contains a coating 30 comprising a carrier material for carrying out a physical separation method.

Accordingly, while only a few embodiments of the present invention have been shown and described, it is obvious that many changes and modifications may be made thereunto without departing from the spirit and scope of the invention.

What is claimed is:

1. A method for determining DNA damage, comprising:
treating cells with genotoxic agents by placing the cells into various chambers of a multi-chamber plate and putting a plurality of genotoxic chemicals into the various chambers of the multi-chamber plate;
lysing the cells; and
separating DNA fragments of the cells according to their length by a physical separating method, wherein walls of the multi-chamber plate are removed and the physical separation process is carried out together for all samples.

2. The method according to claim 1, wherein the step of treating and the step of separating are carried out in the same place.

3. The method according to claim 1, wherein the walls of the multi-chamber plate are dismountable and the step of lysing is carried out after removing the chamber walls.

4. The method according to claim 1, wherein the cells are moved away by a microscope displaceable in at least two dimensions.

5. The method according to claim 4, wherein pictures of the samples are created using the displaceable microscope and are automatically evaluated.

6. The method according to claim 1, wherein a toxicity test is integrated in the method.

7. The method according to claim 1, wherein a toxicity test is carried out on a same cell sample for which the gene damage is measured.

8. A multi-chamber plate for biological, medical, pharmaceutical and chemical research for determining DNA damage, said chamber being used to treat cells with genotoxic agents by placing the cells into various chambers of said plate and putting a plurality of genotoxic chemicals into the various chambers, lysing the cells and separating DNA fragments of the cells according to their length by a physical separating method, the plate comprising:
chamber walls that are removable; and
a coating at least in a bottom area of the plate.

9. The multi-chamber plate according to claim 8, wherein the walls of the multi-chamber plate have a preset breaking point.

10. The multi-chamber plate according to claim 8, wherein the walls of the multi-chamber plate are affixed on the bottom of the multi-chamber plate using an adhesive compound or a sticking compound.

11. The multi-chamber plate according to claim 8, wherein the multi-chamber plate has 96 chambers.

12. The multi-chamber plate according to claim 8, wherein the multi-chamber plate has 8 chambers.

13. The multi-chamber plate according to claim 8, wherein the walls are constructed as non-reflecting.

14. The multi-chamber plate according to claim 8, wherein the multi-chamber plate is coated in the bottom area with a carrier material for carrying out a physical separation method.

15. The multi-chamber plate according to claim 14, wherein the carrier material has gelatin or agarose.

16. The multi-chamber plate according to claim 14, wherein, a base layer, especially agarose is provided underneath the carrier material.

17. The multi-chamber plate according to claim 14, wherein a layer for cell adhesion is provided above the carrier material.

18. The multi-chamber plate according to claim 17, wherein the cell adhesive layer has poly-lysine.
